(19) United States
(12) Patent Application Publication
(10) Pub. No.: US 2007/0274939 A1
(43) Pub. Date: Nov. 29, 2007

(54) Color Cosmetic Compositions

(76) Inventors: Sandra Guerra, Somerset, NJ (US); Ida Marie Sandewicz, Monroe Township, NJ (US); Tatyana Zamyatin, Princeton Juntion, NJ (US); Ivonne Brown, Roosevelt, NY (US)

Correspondence Address: JULIE BLACKBURN;REVLON CONSUMER PRODUCTS CORPORATION, 237 PARK AVENUE, NEW YORK, NY, 10017, US

(21) Appl. No.: 11/420,587

(22) Filed: May 26, 2006

(51) Int. Cl. A61K 8/46; A61K 8/33

(52) U.S. Cl. 424/63; 424/70.31; 424/70.24

(57) ABSTRACT

A color cosmetic composition comprising at least one silicone film forming polymer, at least one pigment, and at least one dispersant that aids in dispersion of the pigment and silicone film forming polymer in the composition.

### TECHNICAL FIELD

The invention is in the field of color cosmetic compositions for application to skin, nails, and hair.

### BACKGROUND OF THE INVENTION

Pigments or colorants are found in nearly all cosmetic products. Color cosmetic products such as blush, eye shadow, mascara, foundation makeup, concealer, and the like, typically contain organic or inorganic pigments or both. In nearly all cases, these pigments cannot be incorporated into the cosmetic composition as it is being made in a cookbook like fashion. Rather, the pigments must be pre-milled, or ground, with a portion of the waxes or oils in the composition. That pre-blend is then added to the composition. This two step operation ensures that the pigments will properly disperse in the composition. If unmilled pigments are added to a cosmetic composition as it is being made, the pigments may not properly disperse. Pre-milling pigments is a time consuming and expensive procedure. The manufacturing facility must usually dedicate a separate room for pigment milling because it is a messy process that would otherwise contaminate the rest of the manufacturing facility. Special milling equipment such as roller mills are needed, and must be cleaned well between each different milling process so that the pigment batches do not contaminate each other. Milling is also time consuming, as plant workers must combine the pigments and waxes or oils, suit up for the milling room, operate the milling equipment to grind the pigments, supervise the process, then make sure the milling equipment is cleaned up for the next user.

While cosmetic formulators use ingredients to promote better dispersion of pigments in a cosmetic formula, these dispersants are not so effective that they eliminate the need for milling pigments. Elimination of pigment dispersion, or at least improving pigment dispersions in cosmetic compositions, is of great interest to cosmetic formulators and manufacturers for obvious reasons.

In addition, it is necessary to heat most color cosmetic compositions in order to properly disperse the ingredients in the composition. The heating takes time and energy. Heat can also cause degradation of heat labile ingredients in the formula. Manufacture of color cosmetic compositions using a “cold process”, that is, where no heat is used, is of obvious interest.

Most unexpectedly, it has been discovered that certain cosmetic ingredients have dispersing properties that are so effective that formulators can eliminate or reduce the pigment milling required in manufacture of color cosmetic products and/or facilitate cold process manufacture of cosmetic compositions.

It is an object of the invention to provide color cosmetic compositions containing certain dispersants that eliminate or greatly reduce the need for milling cosmetic pigments prior to incorporation into formulas and facilitate cold process manufacture.

It is a further object of the invention to provide color cosmetic compositions containing certain improved dispersants.

It is a further object of the invention to provide color cosmetic compositions containing dispersant ingredients that reduce the amount of milling time necessary for pigments.

### SUMMARY OF THE INVENTION

The invention is directed to a color cosmetic composition comprising at least one silicone film forming polymer, at least one pigment, and at least one dispersant that aids in dispersion of the pigment and silicone film forming polymer in the composition.

### DETAILED DESCRIPTION

### I. The Dispersant

A variety of dispersants may be suitable. Preferably, the dispersant is present in amounts ranging from about 0.001-45%, preferably from about 0.005-35%, more preferably from about 0.01-30% by weight of the total composition. In one preferred embodiment of the invention the dispersant is such, and is present in the amount sufficient, to reduce or completely eliminate the need for pigment milling in the preparation of color cosmetic products and/or facilitate manufacture of the cosmetic composition by cold process.

Preferred dispersants are anionic surfactants containing at least one sulfate or sulfonated group. More preferred are dispersants that are fatty acid sulfates or sulfonates. Examples of such surfactants are those listed in the category “cleansing surfactants” on pages 1789 to 1795 of the C.T.F.A. Cosmetic Ingredient Dictionary and Handbook, Eighth Edition, 2000. More preferred are alkali metal or alkaline earth metal fatty acid sulfates or sulfonates, or fatty acid olefin sulfates or sulfonates. The term “fatty acid” when used herein means a saturated or unsaturated, straight or branched chain fatty acid having from about 6 to 40 carbon atoms. Alkali or alkaline earth metals include sodium, potassium, lithium, magnesium, and so on.

Examples of suitable dispersants are sodium fatty acid sulfonates or sulfates including sodium C₁₃₋₁₇ alkane sulfonate, sodium C₁₄₋₁₈ alkane sulfonate, sodium C₉₋₂₂ alkyl sec sulfonate, sodium C₁₂₋₁₃ alkyl sulfate, sodium C₁₂₋₁₅ alkyl sulfate, sodium C₁₂₋₁₅ alkyl sulfate, sodium C₁₂₋₁₈ alkyl sulfate, sodium C₁₆₋₂₀ alkyl sulfate, sodium C14-16 olefin sulfonate, sodium lauryl sulfate, sodium laureth sulfate, sodium oleate, and so on. Particularly preferred is sodium C14-16 olefin sulfonate, which is preferably coated onto the pigments. Such coated pigments can be purchased from Presperse under the trade name Aquaspersibils.

### II. The Silicone Film Former

A variety of silicone film formers may be used in the compositions of the invention. Such film formers are preferably polymeric. The silicone film former may be all silicone, or may be a copolymer of silicone and other organic moieties such as acrylates, methacrylates, and so on. Such film formers may range from about 0.1-60%, preferably from about 0.5-55%, more preferably from about 1 to 45% by weight of the total composition.

A. Silicone Resins

Silicone resins are suitable film forming polymers. Such silicone resins may contain M units and T or Q units or both. The silicone resin may be a liquid, semi-solid, or solid at room temperature.

The term “M” means “monofunctional”, and refers to a siloxy unit that contains one silicon atom bonded to one oxygen atom, with the remaining three substitutents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, the monofunctional siloxy unit is designated by the letter “M”, and means a unit having the general formula:

R₁R₂R₃SiO_{1/2}

wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆); hydroxyl; or hydrogen.

The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

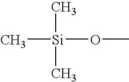

When this monofunctional unit is polymerized with one or more of the other units the oxygen atom will be shared by another silicon atom, i.e. the silicon atom in the monofunctional unit is bonded to ½ of this oxygen atom.

The term “T” in silicone nomenclature means “trifunctional” and refers to a trifunctional siloxy unit.

A “T” unit has the general formula:

R₁SiO_{3/2}

wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula:

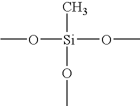

When this trifunctional unit is polymerized with one or more of the other units, the silicon atom shares three oxygen atoms with other silicon atoms, i.e. will share three halves of an oxygen atom.

The term “Q” means “tetrafunctional” with respect to a siloxy unit. A “Q” unit has the general formula:

SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

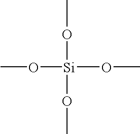

The silicone resin polymers used in the composition of the invention may also contain other units such as “D” units, in addition to the M, T, and Q units described above.

The term “D” in standard silicone nomenclature means “difunctional” with respect to a siloxy unit. If the D unit is substituted with substitutents other than methyl the “D” designation is sometimes used, which indicates a substitutent other than methyl. For purposes of this disclosures a “D” unit has the general formula:

R₁R₂SiO_{2/2}

wherein R₁ and R₂ are defined as above. The SiO_{2/2} designation means that the silicon atom in the difunctional unit is bonded to two oxygen atoms when the unit is polymerized with one or more of the other units. For example, when R₁, R₂, are methyl the resulting difunctional unit is of the formula:

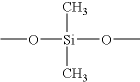

### When this difunctional unit is polymerized with one or more of the other units the silicon atom will be bonded to two oxygen atoms, i.e. will share two one-halves of an oxygen atom.

The silicone resin polymer used in the compositions of the invention may be a combination of M and Q units, a combination of M and T units, a combination of M and Q+T units, or all three of such combinations additionally containing one or more “D” units.

Preferably, the silicone resin polymer used in the compositions of the invention has the INCI name trimethylsiloxysilicate (MQ) or polymethylsilsesquioxane (MT). One type of preferred resin contains M units which are greater than, up to three times greater, than the number of Q units, T units, D units, or combinations thereof, which provides a liquid MQ resin. In one other preferred embodiment, the silicone resin is an MQ resin which is a solid at room temperature and exists in the form of small particulate flakes.

The silicone resin polymers used in the compositions of the invention are made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following:

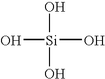

The above hydroxy substituted silane is then condensed or polymerized with other types of silanol substituted units including but not limited to those such as:

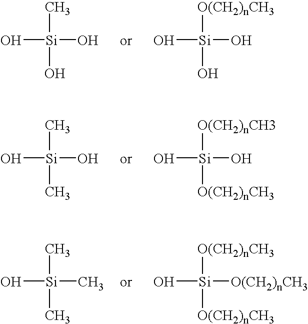

wherein n is 0-10, preferably 0-4.

Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above.

Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

B. Polyoxyalkylene Polydimethylsiloxane Copolymers

While copolymers of polydimethylsiloxane and polyoxyalkylene substituted siloxanes are generally thought of as surfactants, such polymers also provide film forming properties. Such silicone surfactants are generally referred to as dimethicone copolyols or alkyl dimethicone copolyols.

Silicone surfactants typically have at least one hydrophilic radical and at least one lipophilic radical. They may be liquids, solids, or semi-solids at room temperature. They are typically water-in-oil or oil-in-water type surfactants having a Hydrophile/Lipophile Balance (HLB) ranging from about 2 to 18. One preferred silicone surfactant is a nonionic surfactant having an HLB ranging from about 2 to 12, preferably about 2 to 10, most preferably about 4 to 6. The HLB of a nonionic surfactant is the balance between the hydrophilic and lipophilic portions of the surfactant and is calculated according to the following formula:

HLB=7+11.7×log M_{w}/Mₒ

where M_{w} is the molecular weight of the hydrophilic group portion and Mₒ is the molecular weight of the lipophilic group portion.

As used herein the term “silicone surfactant” means an organosiloxane polymer containing a polymeric backbone including repeating siloxy units that may have cyclic, linear or branched repeating units, e.g. di(lower)alkylsiloxy units, preferably dimethylsiloxy units. The hydrophilic portion of the organosiloxane is generally achieved by substitution onto the polymeric backbone of a radical that confers hydrophilic properties to a portion of the molecule.

The hydrophilic radical may be substituted on a terminus of the polymeric organosiloxane, or on any one or more repeating units of the polymer. In general, the repeating dimethylsiloxy units of modified polydimethylsiloxanes are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the polymer, or on any one or more repeating units of the polymer. It should also be understood that the organosiloxane polymer should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof.

The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀ alkyl may be non-interrupted, or interrupted by one or more oxygen atoms, a benzene ring, amides, esters, or other functional groups.

The silicone surfactant may have any of the following general formulas:

MₓQ_{y}, or

MₓT_{y}, or

MDₓD′_{y}D″₂M

wherein each M is independently a substituted or unsubstituted trimethylsiloxy endcap unit. If substituted, one or more of the hydrogens on the endcap methyl groups are substituted, or one or more methyl groups are substituted with a substitutent that is a lipophilic radical, a hydrophilic radical, or mixtures thereof. T is a trifunctional siloxy unit having the empirical formula RSiO_{1.5} or R′SiO_{1.5}. Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:

MDₓD′_{y}D″_{z}M

wherein
M=RRRSiO_{0.5}
D=RRSiO_{1.0}
D′=RR′SiO_{1.0}
D=R′R′SiO_{1.0}
x, y, and z are each independently 0-1000,
where R is methyl or hydrogen, and R′ is a hydrophilic radical or a lipophilic radical, with the proviso that the compound contains at least one hydrophilic radical and at least one lipophilic radical.

### Most preferred is wherein

M=trimethylsiloxy

D=Si[(CH₃)][(CH₂)ₙCH₃]O_{1.0} where n=0-40,

D′=Si[(CH₃)][(CH₂)ₒ—O—PE)]O_{1.0} where PE is (—C₂H₄O)ₐ(—C₃H₆O)_{b}H, o=0-40,

a=1-100 and b=1-100, and

D″=Si (CH₃)₂O_{1.0}

More specifically, suitable silicone surfactants have the formula:

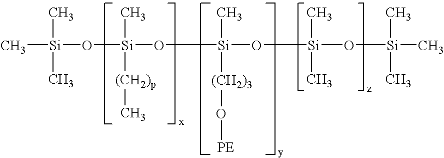

wherein p is 0-40, and

### PE is (—C₂H₄O)ₐ(—C₃H₆O)_{b}—H

where x, y, z, a, and b are such that the maximum molecular weight of the polymer is approximately about 50,000.

Another type of silicone surfactant suitable for use in the compositions of the invention are emulsifiers sold by Union Carbide under the Silwet™ trademark. These surfactants are represented by the following generic formulas:

(Me₃Si)_{y-2}[(OSiMe₂)_{x/y}O—PE]_{y}

wherein

PE=-(EO)ₘ(PO)ₙR

R=lower alkyl or hydrogen

Me=methyl

EO is polyethyleneoxy

PO is polypropyleneoxy

m and n are each independently 1-5000

x and y are each independently 0-5000, and

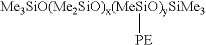

wherein

PE=—CH₂CH₂CH₂O(EO)ₘ(PO)ₙZ

Z=lower alkyl or hydrogen, and

Me, m, n, x, y, EO and PO are as described above,

with the proviso that the molecule contains a lipophilic portion and a hydrophilic portion. Again, the lipophilic portion can be supplied by a sufficient number of methyl groups on the polymer.

As with both types of silicone surfactants, the hydrophilic radical can be substituted on the terminal portions of the silicone, or in other words in the alpha or omega positions or both.

Also suitable as the silicone surfactants are hydroxy-substituted silicones such as dimethiconol, which is defined as a dimethyl silicone substituted with terminal hydroxy groups.

Examples of silicone surfactants are those sold by Dow Corning under the tradename Dow Corning 3225C or 5225C Formulation Aid, Dow Corning 190 Surfactant, Dow Corning 193 Surfactant, Dow Corning Q2-5200, Abil WE97, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the compositions of the invention.

C. Alkyl Silicones

Also suitable are various long chain alky silicones that may be liquids or solids. These are typically also known as silicone waxes. Such alkyl silicones are in generally in the polymeric form and have the formula:

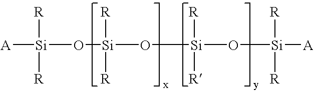

wherein R is methyl and R′ is C₄₋₃₀ alkyl, and x and y are each independently 0-1,000,000 with the proviso that there is at least one x and y, and A is siloxy endcap unit, preferably trimethylsiloxy. Particularly preferred silicones falling within this general formula are cetyl dimethicone, a liquid silicone wax; and stearyl and behenyl dimethicones, both solids at room temperature.

D. Silicone Gums

Also suitable as the silicone film former are various types of silicone gums. The term “silicone gum” means a higher molecular weight silicone polymer that has the texture of a gummy solid. The silicone gum may be diluted or dispersed in liquid silicone oil. Suitable dimethicone gums generally a viscosity of greater than about 500 centistokes, and all the way up to about 90 million centistokes, such viscosity being measured at 25° C. Such silicone gums may be purchased from a variety of silicone suppliers including Dow Corning, under the trade names 1411, 1413, 1418, 1501, and 1503 Fluids. These fluids are blends where the dimethicone gum is solvated or dispersed in a sufficient amount of liquid carrier (such as cyclomethicone, dimethicone) to make the mixture a liquid at room temperature (25° C.). The silicone gum can be dimethicone or dimethiconol, the latter being a hydroxy functional dimethylpolysiloxane gum.

E. Silicone Esters

Suitable silicone film formers include silicone esters set forth in U.S. Pat. No. 5,725,845, which is hereby incorporated by reference in its entirety. Other silicone esters include those comprising units of the general formula RₐR_{b}^{E}SiO_{[4-(a+b)/2]} or Rₓ¹³R_{y}^{E}SiO_{1/2} wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieties (e.g. of about 2, often about 3 to 10 carbon atoms) and one or more aliphatic alcohol moieties (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Silicone esters having the above formula are disclosed in U.S. Pat. No. 4,725,658 and U.S. Pat. No. 5,334,737, which are hereby incorporated by reference. Preferred silicone esters are the liquid siloxy silicates disclosed in U.S. Pat. No. 5,334,737. e.g. diisostearoyl trimethylolpropane siloxysilicate (prepared in Examples 9 and 14 of this patent), and dilauroyl trimethylolpropane siloxy silicate prepared in Example 5 of the patent), which are commercially available from General Electric under the tradenames SF 1318 and SF 1312, respectively.

F. Silicone Elastomers

Suitable silicone film formers may also include cross-linked organosiloxane compounds also known as silicone elastomers. Such elastomers are generally prepared by reacting a dimethyl methylhydrogen siloxane with a crosslinking group comprised of a siloxane having an alkylene group having terminal olefinic unsaturation, or with an organic group having an alpha or omega diene. Such elastomers may also have hydrophilic groups such as ethylene oxide or, glyceryl groups, or propylene oxide. Examples of suitable silicone elastomers for use as thixotropic agents include Dow Corning 9040, sold by Dow Corning, and various elastomeric silicones sold by Shin-Etsu under the KSG tradename including KSG 15, KSG 16, KSG 19, KSG 21, KSG 710, and so on.

G. Copolymers of Silicones and Ethylenically Unsaturated Monomers

Another type of film forming polymer that may be used in the compositions of the invention is obtained by reacting silicone moieties with ethylenically Unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula:

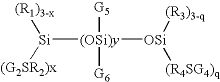

wherein G₅ represents monovalent moieties which can independently be the same or different selected from the group consisting of alkyl aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z is methylene or propylene.

G₆ is a monovalent moiety, which can independently be the same or different selected from the group consisting of alkyl, aryl, aralkyl, alkoxy, alkylamino, fluoroalkyl, hydrogen, and -ZSA;

G₂ comprises A;

G₄ comprises A;

R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.

R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and

R₃ is a monovalent moiety, which is independently alkyl, aryl, aralkyl, alkoxy, alkylamino, fluoroalkyl, hydrogen, or hydroxyl, preferably C₁₋₄ alkyl or hydroxyl, most preferably methyl;

R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene.

x is an integer of 0-3,

y is an integer of 5 or greater; preferably 10 to 270, and more preferably 40-270; and

q is an integer of 0-3.

These polymers are described in U.S. Pat. No. 5,468,477, which is hereby incorporated by reference. Most preferred is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved or dispersed in one or more of the liquids that may be found in the composition such as volatile oils (isododecane), water, or other non-volatile or volatile oils. Preferred is where the polymer is in dry particulate form, and as such it can be dissolved in one or more of the liquids comprising the liquid carrier. This polymer has the CTFA name Polysilicone-6.

Another type of such a polymer comprises a vinyl, methacrylic, or acrylic backbone with pendant siloxane groups and pendant fluorochemical groups. Such polymers preferably comprise comprise repeating A, C, D and optionally B monomers wherein:

A is at least one free radically polymerizable acrylic or methacrylic ester of a 1,1-dihydroperfluoroalkanol or analog thereof, omega-hydridofluoroalkanols, fluoroalkylsulfonamido alcohols, cyclic fluoroalkyl alcohols, and fluoroether alcohols,

B is at least one reinforcing monomer copolymerizable with A,

C is a monomer having the general formula X(Y)nSi(R)₃-m Z.m wherein

X is a vinyl group copolymerizable with the A and B monomers,

Y is a divalent linking group which is alkylene, arylene, alkarylene, and aralkylene of 1 to 30 carbon atoms which may incorporate ester amide, urethane, or urea groups,

n is zero or 1;

m is an integer of from 1 to 3,

R is hydrogen, C₁₋₄ alky, aryl, or alkoxy,

Z is a monovalent siloxane polymeric moiety; and

D is at least one free radically polymerizable acrylate or methacrylate copolymer.

Such polymers and their manufacture are disclosed in U.S. Pat. Nos. 5,209,924 and 4,972,037, which are hereby incorporated by reference. One type of such a polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. Most preferred is a polymer having moieties of the general formula:

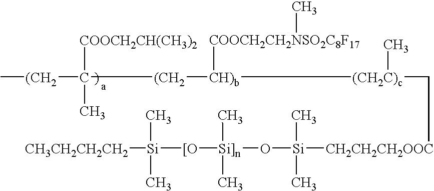

wherein each of a, b, c, and n have a value in the range of 1-100,000, and the terminal groups are selected from the group consisting of a C₁₋₂₀ straight or branched chain alkyl aryl, and alkoxy and the like. These polymers may be purchased from Minnesota Mining and Manufacturing Company under the tradenames of Silicone Plus polymers. Most preferred is poly(isobutyl methacrylate -co- methyl FOSEA) -g- poly(dimethylsiloxane) which is sold under the tradename SA 70-5 IBMMF or Polysilicone 7.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:

A is at least on free radically polymerizable vinyl, methacrylate, or acrylate monomer;

B, when present, is at least one reinforcing monomer copolymerizable with A,

C is a monomer having the general formula:

X(Y)ₙSi(R)₃₋ₘZₘ

wherein:

X is a vinyl group copolymerizable with the A and B monomers;

Y is a divalent linking group;

n is zero or 1;

m is an integer of from 1 to 3;

R is hydrogen, C₁₋₁₀ alkyl, substituted or unsubstituted phenyl, C₁₋₁₀ alkoxy; and

Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609. Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 5,618,524, issued Apr. 8, 1997, all of which are incorporated by reference herein in their entirety.

H. Copolymers of Silicones and Urethanes

Also suitable as the film forming polymer are copolymers of silicones and urethane moieties, also referred to as silicone urethanes. Urethanes are generally formed by the reaction of polyhydroxy compounds with diisocyanates, as follows:

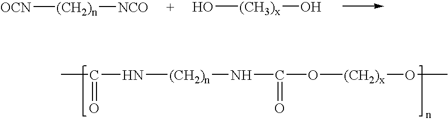

wherein x is 1-1000.

I. Copolymers of Silicones and Amides

Another type of silicone film forming copolymer includes polymers referred to as silicone polyamides. Such polymers generally comprise silicone moieties that are reacted with amide moieties, such as those having the general formula:

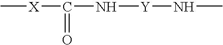

wherein X and Y are each independently linear or branched alkylene having 1-40 carbon atoms, which may be substituted with one or more amide, hydrogen, alkyl, aryl, or halogen substitutents.

Suitable silicone polyamides are set forth in U.S. Patent Publication No. 2004/0180032A1 which is hereby incorporated by reference in its entirety.

### III. Particulates

The composition also contains particulates, which may be in the form of pigments, powders, and the like. Such particulates may be present ranging from about 0.1-75%, preferably from about 0.5-65%, more preferably from about 1-50% by weight of the total composition. In the case where the composition may comprise mixtures of pigments and powders, suitable ranges include about 0.01-75% pigment and 0.1-75% powder, such weights by weight of the total composition.

A. Powders

The particulate matter may be colored or non-colored (for example white) non-pigmentitious powders. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

B. Pigments

The particulate materials may comprise various organic and/or inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable

### IV. Other Ingredients

The compositions of the invention may be in an anhydrous, emulsion, or solution form. If in the emulsion form, from about 0.1-99% water and from about 0.1-99% oil are acceptable.

A. Oils

Whether in the emulsion or anhydrous form, the compositions of the invention may comprise one or more oils. The term “oil” in the context of this invention means an animal, vegetable, mineral, synthetic, or silicone oil that is liquid or semi-solid at room temperature. The oil may be volatile or non-volatile. The term “volatile” means that the oil has a vapor pressure of greater than about 2 mm. of mercury at 20° C. The term “non-volatile” means that the oil has a vapor pressure of less than about 2 mm. of mercury at 20° C. If present, suggested ranges of oil found in the compositions of the invention are from about 0.1-80%, preferably about 0.5-75%, more preferably about 1-70% by weight of the total composition. Examples of oils suitable for use in the composition include, but are not limited to those set forth herein.

(1). Silicone Oils

Suitable silicone oils include volatile linear or cyclic silicones. Generally such silicones have a viscosity ranging from about 0.1 to 10 centistokes at 25° C. If present, suggested ranges of volatile silicone are from about 0.1-80%, preferably about 0.5-75%, more preferably about 1-65% by weight of the total composition.

Cyclic silicones (or cyclomethicones) are of the general formula:

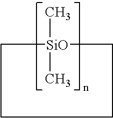

where n=3-6.

Linear volatile silicones that may be used in the compositions of the invention generally having the formula:

(CH₃)₃Si—O—[Si(CH₃)₂—O]ₙ—Si(CH₃)₃

where n=0-7, preferably 0-5, more preferably 1-4. Examples of such linear volatile silicones include hexamethyldisiloxane, octamethyltrisiloxane, decamethyltetrasiloxane, dodecamethylpentasiloxane, and mixtures thereof.

Linear and cyclic volatile silicones are available from various commercial sources including Dow Corning Corporation and General Electric. The Dow Corning volatile silicones are sold under the trade names Dow Corning 244, 245, 344, and 200 fluids. These fluids comprise octamethylcyclotetrasiloxane, decamethylcyclopentasiloxane, cyclohexasiloxane, and mixtures thereof.

Also suitable for use in the compositions of the invention are various non-volatile silicone oils, both water soluble and water insoluble. Such silicones preferably have a viscosity ranging from about 5 to 499,000 centipoise, preferably 10 to 350,000 centipoise at 25° C. Suitable water insoluble silicones include amine functional silicones such as amodimethicone; phenyl substituted silicones such as phenyl trimethicone, phenyl dimethicone, dimethicone, and the like. These types of silicone oils are available from a variety of sources including Dow Corning Corporation, GE Silicones, Wacker, and the like.

(2). Hydrocarbons

The oil may comprise one or more volatile or non-volatile hydrocarbon oils. Examples of volatile hydrocarbons include various straight or branched chain paraffinic hydrocarbons having 5 to 40 carbon atoms, more preferably 8-16 carbon atoms. Suitable hydrocarbons include pentane, hexane, heptane, octane, decane, dodecane, tetradecane, tridecane, and C₈₋₂₀ isoparaffins such as isododecane, isohexadecane, and those disclosed in U.S. Pat. Nos. 3,439,088 and 3,818,105, both of which are hereby incorporated by reference. Preferred volatile paraffinic hydrocarbons have a molecular weight of about 70-225, preferably about 160 to 190 and a boiling point range of 30 to 320°, preferably 60-260° C., and a viscosity of less than about 10 centipoise at 25° C. Such paraffinic hydrocarbons are available from EXXON under the ISOPARS trademark, and from the Permethyl Corporation.

Suitable nonvolatile hydrocarbon oils include longer chain isoparaffins and olefins, preferably those having greater than about 18 to 20 carbon atoms. Examples of such hydrocarbon oils include C₂₄₋₂₈ olefins, C₃₀₋₄₅ olefins, C₂₀₋₄₀ isoparaffins; polyisobutene, polydecene, polybutene, and hydrogenated derivatives thereof, mineral oil, pentahydrosqualene, squalene, squalane, and mixtures thereof.

Also suitable are lower organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety. Such silicones or organic oils include those further described as follows.

(3). Esters

Suitable esters that may be used in the compositions of the invention are mono-, di-, and triesters. The composition may comprise one or more esters selected from the group, or mixtures thereof.

(a). Monoesters

Monoesters are defined as esters formed by the reaction of a monocarboxylic acid having the formula R—COOH, wherein R is a straight or branched chain saturated or unsaturated alkyl having 2 to 50 carbon atoms, or phenyl; and an alcohol having the formula R—OH wherein R is a straight or branched chain saturated or unsaturated alkyl having 2-50 carbon atoms, or phenyl. Both the alcohol and the acid may be substituted with one or more hydroxyl groups, or may contain other groups such as ester, ether, and the like. Either one or both of the acid or alcohol may be a “fatty” acid or alcohol, and may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, cetyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(b). Diesters

Suitable diesters that may be used in the compositions of the invention are formed by the reaction of a dicarboxylic acid and an aliphatic or aromatic alcohol, or the reaction of an aliphatic or aromatic alcohol having at least two hydroxyl groups with one or more carboxylic acids. The dicarboxylic acid may contain from 2 to 50 carbon atoms, and may be in the straight or branched chain, saturated or unsaturated form. The dicarboxylic acid may be substituted with one or more hydroxyl groups. The aliphatic or aromatic alcohol may also contain 2 to 50 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, esters of neopentyl glycol such as neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(c). Triesters

Suitable triesters comprise the reaction product of a tricarboxylic acid and an aliphatic or aromatic alcohol, or alternatively, the reaction of an aliphatic or aromatic alcohol having at least three hydroxyl groups with one or more carboxylic acids. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, and so on.

(d). Tetraesters

Suitable tetraesters comprise the reaction product of alcohols having four hydroxyl groups such as pentaerythritol, with carboxylic acids which may be the same or different, and as described above with respect to the mono-, di-, and triesters. Examples of such tetraesters include esters of pentaerythritol and C₁₋₃₀ monocarboxylic acids. All of the hydroxyl groups may be reacted with monocarboxylic acids, or only one, two, or three.

(4). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(5). Fluorinated Oils

Also suitable as for use in the composition are various fluorinated oils such as fluorinated silicones, fluorinated esters, or perfluoropolyether. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference.

Perfluoropolyethers like those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester that is formed by the reaction of a guerbet alcohol having the general formula:

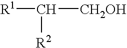

and a fluoroalcohol having the following general formula:

CF₃—(CF₂)ₙ—CH₂—CH₂—OH

wherein n is from 3 to 40.

with a carboxylic acid having the general formula:

R³COOH, or

HOOC—R³—COOH

wherein R¹, R², and R³ are each independently a straight or branched chain alkyl.

The guerbet ester may be a fluoro-guerbet ester, which is formed by the reaction of a guerbet alcohol and carboxylic acid (as defined above), and a fluoroalcohol having the following general formula:

CF₃—(CF₂)ₙ—CH₂—CH₂—OH

wherein n is from 3 to 40.

Examples of suitable fluoro guerbet esters are set forth in U.S. Pat. No. 5,488,121, which is hereby incorporated by reference. Suitable fluoro-guerbet esters are also set forth in U.S. Pat. No. 5,312,968, which is hereby incorporated by reference.

B. Natural or Synthetic Waxes

A variety of waxes may be used in the compositions of the invention including animal, vegetable, mineral, or silicone waxes. If present in the composition, the waxes may range from about 0.1-50%, preferably about 0.5-40%, more preferably about 1-38% by weight of the total composition. Generally such waxes have a melting point ranging from about 28 to 125° C., preferably about 30 to 100° C. Examples of animal, vegetable, or mineral waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of ethylene homo- or copolymeric waxes such as polyethylene (also referred to as synthetic wax), polypropylene, and mixtures thereof.

Also suitable are various types of silicone waxes, referred to as alkyl silicones, which are polymers that comprise repeating dimethylsiloxy units in combination with one or more methyl-long chain (C₁₆₋₃₀) alkyl units where the long chain alkyl is preferably a fatty chain that provides a wax-like characteristic to the silicone. Such silicones include, but are not limited to stearoxydimethicone, behenoxy dimethicone, stearyl dimethicone, cetearyl dimethicone, cetyl dimethicone, and so on. Suitable waxes are set forth in U.S. Pat. No. 5,725,845, which is hereby incorporated by reference in its entirety.

C. Rheological Additives

The compositions of the invention may comprise one or more rheological additives. The term “rheological additive” means an ingredient or combination of ingredients that increase the viscosity of, or thicken, the composition, and if particulates are present, may also suspend the particulates in the composition. If a rheological additive is present, most desired is one that is a non-matting rheological additive, which means that it exhibits a reduced tendency to mute or matte the shininess of the silicone resin polymer. Suggested ranges of rheological additive are from about 0.01-60%, preferably about 0.05-50%, more preferably about 0.1-45% by weight of the total composition.

One type of non-matting rheological additive comprises natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, bentones, and the like. Another example of such a rheological additive is silicate metal silicate gelling agents, such as those sold under the tradename Laponite®.

Also suitable as rheological additives are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonylphenyl

Another type of rheological additive that may be used in the compositions are silicas, silicates, silica silylate, and derivatives thereof. These silicas and silicates are generally found in the particulate form.

D. Plasticizers

It may also be desirable to include plasticizers in the compositions of the invention. Plasticizers may improve the spreadability and application of the composition to the surface to which it is applied and in some cases will interact with the film forming polymer to make it more flexible. If present, the plasticizer may be found in the oil or water phase if the composition of the invention is in the form of an emulsion, and in the oil or lipophilic phase if the composition is in the anhydrous form. A variety of plasticizers are suitable including Suitable plasticizers include glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like. Also suitable, are plasticizers of the following general formula:

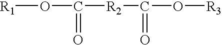

wherein R₁, R₂, and R₃ are each independently a C₁₋₂₀ straight or branched chain alkyl or alkylene which may be substituted with one or more hydroxyl groups. Preferably, R₁ is a C₃₋₁₀ straight or branched chain alkyl; R₂ is a C₂₋₈ alkyl that may be substituted with one or more hydroxyl groups; and R₃ is a C₃₋₁₀ straight or branched chain alkyl. Examples of such compounds include dioctyl malate, diisopropyl adipate, dibutyl adipate, dibutyl sebacate, dioactyl azelate, dioctyl succinate, dioctyl fumarate, and the like.

E. Non-Silicone Film Forming Polymers

(1). Synthetic Organic Polymers

Suitable as additional film formers are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

In some cases, polymers containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups are particularly suitable. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula:

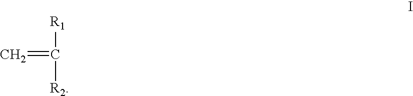

wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM or OCOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

More specific examples include the monofunctional ethylenically unsaturated monomer is of Formula I, above, wherein R₁ is H or a C₁₋₃₀ alkyl, and R₂ is COOM or OCOM wherein M is a C₁₋₃₀ straight or branched chain alkyl which may be substituted with one or more hydroxy groups.

Further examples include where R₁ is H or C₃, and R₂ is COOM wherein M is a C₁₋₁₀ straight or branched chain alkyl, which may be substituted with one or more hydroxy groups.

Di-, tri- and polyfunctional monomers, as well as oligomers, of the above monofunctional monomers may also be used to form the polymer. Suitable difunctional monomers include those having the general formula:

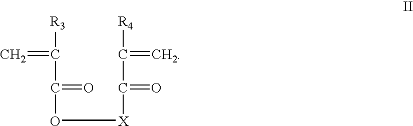

wherein R₃ and R₄ are each independently H, a C₁₋₃₀ straight or branched chain alky, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822.859; all of which are hereby incorporated by reference.

Also suitable are polymers formed from the monomer of Formula I, above, which are cyclized, in particular, cycloalkylacrylate polymers or copolymers having the following general formulas:

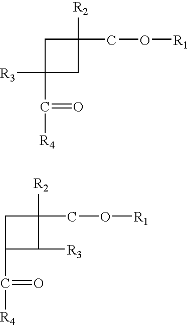

wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane. The monomers mentioned herein can be polymerized with various types of organic groups such as propylene glycol, isocyanates, amides, etc.

Another type of synthetic organic polymer that may be used in the compositions of the invention is obtained by polymerizing ethylenically unsaturated monomers which comprise vinyl ester groups either alone or in combination with other monomers including silicon monomers, other ethylenically unsaturated monomers, or organic groups such as amides, urethanes, glycols, and the like. The various types of monomers or moieties may be incorporated into the film forming polymer by way of free radical polymerization, addition polymerization, or by formation of grafts and blocks which are attached to the growing polymer chain according to processes known in the art.

Typically, this type of film forming polymer comprises vinyl ester monomers having the following general formula:

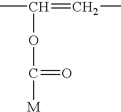

wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substitutents include hydroxyl, ethoxy, amide or amine, halogen, alkyloxy, alkyloxycarbonyl, and the like. Preferably, M is H or a straight or branched chain alkyl having from 1 to 30 carbon atoms. The film forming polymer may be a homopolymer or copolymer having the vinyl ester monomers either alone or in combination with other ethylenically unsaturated monomers, organic groups, or silicon monomers.

Also suitable are various types of organic groups that may be polymerized with the vinyl ester monomers including but not limited to urethane, amide, polyalkylene glycols, and the like as set forth above.

The vinyl ester monomers may also be copolymerized with other ethylenically unsaturated monomers that are not vinyl esters, including those set forth above.

(2). Natural Polymers

Also suitable for use are one or more naturally occurring polymeric materials such as resinous plant extracts including such as rosin, shellac, chitin, and the like.

F. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

G. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition. Vitamins D and K are suitable.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

H. Surfactants

The composition may also contain one or more surfactants in addition to the dispersant. If present, such surfactants may range from about 0.001-30%, preferably from about 0.05-25%, more preferably from about 0.1-20% by weight of the total composition.

Preferred are anionic surfactants such as alkoxylated alcohols, more preferably C6-45 fatty elkoxylated alcohols such as laureth, steareth, ceteareth, beheneth, and so on, where the number of repeating ethoxy units ranges from about 3 to 200. Particularly preferred is where the ethoxylated alcohol is laureth.

### IV. The Compositions

The compositions of the invention may be found in a variety of forms including, but not limited to, creams, lotions, gels, and colored cosmetic compositions such as foundation, lipstick, eyeshadow, blush, concealer, eyeliner, mascara, nail enamel, and the like. Typical ranges of ingredients found in such compositions include, but are not limited to, those set forth herein.

Creams and lotions Generally comprise from about 0.1-99% water, 0.1-99% oil, about 0.001-20% of one or more surfactants, and may optionally include any one or more of the ingredients set forth herein. Creams have a more viscous consistency while lotions tend to be less viscous, or more pourable.

Typical foundation makeup compositions and concealers may be found in the emulsion form and will generally comprise from about 0.1-99% water, 0.1-99% oil, about 0.001-20% of one or more surfactants, and from about 0.01-30% of particulate material which may be pigments, powders, or mixtures thereof. The foundation makeup composition may optionally comprise any of the other ingredients described herein, and in the ranges set forth.

Foundation makeup, powder, and concealer compositions may also be in the anhydrous form. If so, typical ranges of ingredients include from about 0.1-75% oil and about 0.1-25% particulate materials, which may be pigments, powders, or mixtures thereof. Such compositions may optionally contain one or more of the ingredients set forth herein and in the ranges set forth.

Blushes and eyeshadows may be in the water and emulsion form, and if so, typically contain the ranges of ingredients set forth above with respect to foundation makeup and, optionally, any one or more of the other ingredients set forth herein, and in the same amounts. However, blushes and eyeshadows may also be in the anhydrous form and, if so, contain the ranges of ingredients set forth with respect to the anhydrous foundation and powder compositions mentioned above and the optional ingredients listed herein.

Typically, lipsticks contain from about 0.01-99% oil, 0.1-50% structuring agent, and from about 0.1-50% of particulates which may be pigments, powders, or mixtures thereof. The lipsticks may contain one or more of the ingredients mentioned herein and in the same ranges as set forth therein.

Mascara compositions may be in the emulsion form, and if so, typically contain from about 0.1-99% water and from about 0.1-99% oil, and 0.1-50% particulate matter. Optionally, mascaras may contain from about 0.1-50% surfactants, and the other ingredients set forth herein. Mascaras may also be anhydrous, and if so, may comprise from about 0.1-99% oil, 0.1-50% particulate matter, and, optionally, one or more of the ingredients set forth above.

In one embodiment of the invention, the composition may develop color as it is applied to skin. More specifically, if the preferred anionic surfactant treated pigments used in the composition are suspended in the lipophilic phase of the composition when it is stored in the container, the composition may be a pale white or muted color. However, when the composition is applied to the skin the pigments dispersed in the lipophilic phase will migrate into the aqueous phase and develop color as the composition is being applied to the skin. The color development may be even more pronounced when the composition contains very microfine particle size powders than mute the color. The invention includes compositions that develop color on the skin as described in U.S. patent application Ser. Nos. 11/378,827, filed Mar. 17, 2006, entitled Dry Water Cosmetic Compositions That Change Color Upon Application; 11/326,150, filed Jan. 5, 2006, entitled Powdered Water Cosmetic Compositions and Related Methods, 11/381,138, filed Mar. 17, 2006, entitled Makeup Compositions and Methods; 11/378,681, filed Mar. 17, 2006, entitled Color Cosmetic Compositions and Methods; 10/914,571, filed Jul. 12, 2004, entitled Packaged Cosmetic Compositions and Related Methods; and 11/249,882, filed Oct. 13, 2005, entitled Color Cosmetic Compositions; all of which are hereby incorporated by reference in their entirety.

The invention will be further described in connection with the following examples, which are set forth for the purpose of illustration only.

### EXAMPLE 1

A foundation makeup was made as follows:

| | | % by |
|---|---|---|
| | Ingredient | weight |
| 1 | Water | QS |
| 1 | Dipropylene glycol | 7.00 |
| 1 | Magnesium aluminum silicate | 0.30 |
| 1 | Cellulose gum | 0.20 |
| 1 | Xanthan gum | 0.10 |
| 1 | Methyl paraben | 0.20 |
| 2 | PEG-12 dimethicone¹ | |
| 2 | Water | 13.45 |
| 2 | Trisodium EDTA | 0.05 |
| 3 | Cyclopentasiloxane, dimethiconol, laureth-4, laureth-23² | 25.00 |
| 3 | Titanium dioxide, C14-16 olefin sulfonate | 5.00 |
| 3 | Talc, C14-16 olefin sulfonate | 2.85 |
| 3 | Yellow iron oxide, C14-16 olefin sulfonate | 0.85 |
| 3 | Red iron oxide, C14-16 olefin sulfonate | 0.55 |
| 3 | Black iron oxide, C14-16 olefin sulfonate | 0.20 |
| 3 | Boron nitride | 1.50 |
| 3 | HDI/trimethylolhexyllactone crosspolymer | 2.50 |
| 4 | Tocopheryl acetate | 0.05 |
| 4 | Retinyl palmitate | 0.05 |
| 4 | Phenoxyethanol | 1.00 |

¹Dow Corning 5329
²Dow Corning 7-3100 HIP Emulsion

The Sequence 2 ingredients were premixed, then added to Sequence 1. The mixture was homogenized for ten minutes until dispersed, with dispersion checked on a glass slide. Sequence 3 was premixed and added to the mixture of Sequences 1 and 2 and homogenized until dispersed. Sequence 4 was added and mixed with a homo mixer for 20 minutes or until dispersed. Sequence 5 was added and homo mixed at 45 rpm for 15 minutes. Sequence 6 was added and mixed until dispersed. No heat was used.

### EXAMPLE 2

A foundation makeup composition was made as follows:

| | | % by |
|---|---|---|
| Seq | Ingredient | weight |
| 1 | Water | QS |
| 1 | Butylene glycol | 3.00 |
| 1 | 1,2 hexanediol, caprylyl glycol | 0.50 |
| 1 | Pentylene glycol | 3.50 |
| 1 | Red iron oxides, sodium C14-16 olefin sulfonate¹ | 0.29 |
| 1 | Black iron oxide, sodium C14-16 olefin sulfonate² | 0.62 |
| 1 | Yellow iron oxides, sodium C14-16 olefin sulfonate³ | 0.10 |
| 1 | Titanium dioxide, sodium C14-16 olefin sulfonate | 6.00 |
| 1 | Mica | 3.29 |
| 1 | Silica | 2.00 |
| 1 | HDI/trimethylol hexyllactone crosspolymer, silica | 3.00 |
| 1 | Kaolin | 0.50 |
| 1 | PEG-11 methyl ether dimethicone⁴ | 1.00 |
| 2 | Mica, titanium dioxide, iron oxides, silica | 0.50 |
| 3 | Acrylates/C10-30 alkyl acrylate crosspolymer | 0.20 |
| 4 | Neopentylglycol diethylhexanoate | 6.00 |
| 4 | Dimethicone, 10 cs | 8.50 |
| 4 | Lauryl polyglyceryl-3 polydimethylsiloxyethyl | 0.50 |
| | dimethicone⁵ | |
| 4 | Cyclomethicone | 8.00 |
| 4 | Trisiloxane, dimethicone | 5.00 |
| 4 | Cyclomethicone, dimethicone | 3.00 |
| 5 | Water | 3.00 |
| 5 | Triethanolamine | 0.25 |
| 6 | Water, butylene glycol, algin, sodium citrate, | 2.00 |
| | atelocollagen, serine⁶ | |
| 6 | Hydrolyzed viola tricolor extract | 0.50 |
| 7 | Polyacrylamide, C13-14 isoparaffin, laureth-7 | 0.50 |

¹Aquaspersibils, Red Iron Oxide. Presperse Incorporated.
²Aquaspersibils, Black Iron Oxide. Presperse Incorporated.
³Aquaspersibils, Yellow Iron Oxide. Presperse Incorporated.
⁴KF-6011. Shin-Etsu Silicones.
⁵KF-6015. Shin-Etsu Silicones.
⁶Moisturizing Marine MicroPatch ®, Coletica.

The composition was prepared by combining the Sequence 1 ingredients in a beaker and mixing on homomixer at 25° C. until pigments were dispersed. Once the pigments were dispersed in the Sequence 1 mixture, the Sequence 2 ingredients were added to the Sequence 1 ingredients while mixing at low speed. Once the Sequence 2 ingredients were dispersed in the composition the Sequence 3 ingredients were added. The mixing was continued until the batch flowed easily through the mill and the composition looked uniform. The Sequence 4 ingredients were combined and added to the Sequence 3 ingredients with mixing. The Sequence 5 ingredients were then added. The composition was mixed well and poured into containers. No heat was used.

While the invention has been described in connection with the preferred embodiment, it is not intended to limit the scope of the invention to the particular form set forth but on the contrary, it is intended to cover such alternatives, modifications, and equivalents as may be included within the spirit and scope of the invention as defined by the appended claims.

1. A color cosmetic composition comprising at least one silicone film forming polymer, at least one pigment, and at least one dispersant that aids in dispersion of the pigment and silicone film forming polymer in the composition.

2. The composition of claim 1 wherein the dispersant comprises an anionic surfactant.

3. The composition of claim 2 wherein the dispersant comprises a fatty acid ester of a sulfonate or sulfate surfactant.

4. The composition of claim 3 wherein the dispersant comprises a C₆₋₄₀ saturated or unsaturated, straight or branched chain, fatty acid ester of a sulfonate or sulfate.

5. The composition of claim 4 wherein the fatty acid ester is an ester of an olefin sulfate or olefin sulfonate.

6. The composition of claim 5 wherein the fatty acid ester is sodium C₁₄₋₁₆ olefin sulfonate.

7. The composition of claim 6 wherein the fatty acid ester is coated onto the surface of at least one of the pigments or powders found in the composition.

8. The composition of claim 7 wherein the fatty acid ester is coated onto at least one of the pigments found in the composition.

9. The composition of claim 1 further comprising one or more nonionic surfactants.

10. The composition of claim 9 wherein the nonionic surfactant is a alkoxylated fatty alcohol.

11. The composition of claim 10 wherein the alkoxylated fatty alcohol comprises an ethoxylated fatty alcohol.

12. The composition of claim 11 wherein the ethoxylated fatty alcohol is laureth having a number of repeating ethoxy units ranging from 2 to 50.

12. The composition of claim 12 wherein the number of repeating alkoxy groups ranges from 2 to 25.

12. The composition of claim 1 further comprising at least one silicone gum.

13. The composition of claim 12 wherein the silicone gum is dimethiconol.

14. The composition of claim 1 wherein the silicone film former is a silicone resin.

15. The composition of claim 14 wherein the silicone film former is an MQ or MT resin.

16. The composition of claim 1 further comprising at least one monoester, diester, or triester.

17. The composition of claim 16 further comprising at least one volatile silicone or paraffinic hydrocarbon.

18. The composition of claim 1 in the for of a water and oil emulsion.

19. The composition of claim 18 wherein the oil comprises a silicone oil.

20. The composition of claim 18 wherein the emulsion is a water in oil emulsion.
